(19) United States
(12) Patent Application Publication
(10) Pub. No.: US 2020/0140799 A1
(43) Pub. Date: May 7, 2020

(54) Bioreactors with Filters

(71) Applicant: GE Healthcare Bio-Sciences Corp., Marlborough, MA (US)

(72) Inventor: Yasser S. Ali, Westborough, MA (US)

(21) Appl. No.: 16/625,173

(22) PCT Filed: Jun. 26, 2018
(86) PCT No.: PCT/EP2018/000318

§ 371 Date: Dec. 20, 2019

Related U.S. Application Data
(60) Provisional application No. 62/524,810, filed on Jun. 26, 2017.

(51) Int. Cl. C12M 1/00; C12M 3/06; C12M 3/00; C12M 1/09

(52) U.S. Cl. CPC C12M 23/14; C12M 29/04; C12M 27/16; C12M 23/56; C12M 29/10; C12M 23/46; C12M 23/26

(57) ABSTRACT

The invention discloses a bioreactor 10 for cell culture and expansion, comprising a bioreactor chamber 17, a filter 13 disposed within the chamber 17 and at least one tether 14 loosely tethering the filter to the bioreactor chamber. In an embodiment, the tether(s) allow the filter 13 to move from side to side and/or up and down within the chamber 17, preferably without touching an inner surface of the bioreactor chamber 17. Alternatives show, two, three or four tethers, but one or more tethers can be used.

### TECHNICAL FIELD

The present invention relates to bioreactors with one or more filters disposed within them.

### BACKGROUND

Cell culture for producing regenerative medicine is done with the aim of harvesting cells which can subsequently be injected into a patient. Hence, health and viability of the cells is of paramount importance. To achieve that, the cells need to be reproduced under controlled conditions and fed with nutrients to grow. One commercially successful disposable bioreactor systems uses a flexible bioreactor placed on a rockable platform. The bioreactor is partially filled with liquid cell culture medium and cells of interest are introduced in the bioreactor. The bioreactor is then placed on the rockable platform and rocked. Rocking of the platform induces wave like motion in the cell culture medium which causes the cells to move around constantly. This also facilitates efficient gaseous exchange between the internal and the external space of the bioreactor.

One way to grow cells is in a perfusion bioreactor where the cells in the bioreactor are fed continuously with fresh cell culture medium to replace the spent cell culture medium while keeping the volume of the cell culture medium constant in the bioreactor. The cells reach a steady state of reproduction and can be maintained in that state for a few weeks until the required cell density is achieved. Perfusion bioreactors typically contain a filter which filters out the spent cell culture medium and toxic cell metabolites that inhibit cell growth out of the bioreactor while retaining the healthy and viable cells within the bioreactor.

One type of perfusion bioreactor is disclosed in U.S. Pat. No. 9,017,997B2, where the filter is fixed to the inner surface of a wall of the bioreactor and thus does not float on the surface of the culture medium. This placement of the filter prevents the filter from getting damaged due to twisting or sticking to the walls of the bioreactor. However, the filter can get easily clogged and fouled as it is fixed to the bottom of the bioreactor. Similarly, WO2012/158108A1 discloses a perfusion bioreactor for cultivation of cells on microcarriers where the filter is fixed to the inner surface of a wall of the bioreactor. Further, WO2015/034416A1 discloses a bioreactor with internal dialysis modules suitable for dialysis cultivation of cells. The dialysis compartments are formed a freely movable bundle of hollow fiber membranes, a pouch attached to an inner wall of the bag, a pouch freely moving or a sheet of membrane fixed to an inner wall of the bag.

Another perfusion bioreactor is disclosed in WO2017/055059A1, where the filter is held by a filter holding device. The filter holding device is attached to an inner wall of the bioreactor such that there is a space between the filter and the inner wall of the bioreactor and the liquid medium provided in the bioreactor can flow on both sides of the filter. This arrangement of the filter holding device although causes a cross flow filtration effect, it is quite limited in reducing the clogging and fouling of the filter due to limited space between the filter and the inner wall of the bioreactor.

In the perfusion bioreactor disclosed in U.S. Pat. No. 6,544,788, the filter is constructed to move freely on the liquid cell culture medium during the rocking motion of the bioreactor. The filter is flicked across the surface of the cell culture medium because of the rocking motion of the bioreactor. This keeps the filter from clogging due to the erosion of any debris by the turbulence generated by the tangential motion of the filter relative to the cell culture medium. However, as the filter is constructed to move freely, it can lead to twisting and turning of the filter thereby damaging it. This design can also cause the filter to stick to the inner walls of the bioreactor thus impairing the gaseous exchange and filtration of the cell culture medium. Also, when the filter is floating on the surface of the cell culture medium, the prefusion process may fail due to the “Bubbling” of the media. When the filter is not fully exposed to the cell culture medium, the pump sucks air instead of the cell culture medium causing the waste collection bag to inflate with air instead of spent cell culture medium. This phenomenon is called bubbling.

### SUMMARY OF THE INVENTION

An object of embodiments of the invention is to provide a bioreactor with a filter system where the filter has one or more tethers that function as floating constraints to enhance the performance of the bioreactor.

One advantage of that embodiment is that the tethers keep the filter immersed in the liquid cell culture media for efficient gaseous exchange and filtration of the cell culture media by preventing the filter from twisting or turning around along an axis which could inhibit the filtration and gaseous exchange.

Another advantage of that embodiment is that the filter is prevented from clogging and fouling easily as the tethers allow the filter to move about constantly when the bioreactor is placed on a rocking platform. Any cells or debris deposited on the filter is eroded by the tangential flow of the cell culture media with respect to the filter.

Another advantage of that embodiment is that the tethers keep the filter exposed to the cell culture media always while moving and thus prevents bubbling of the media.

According to an embodiment of the invention, the tethers on the filter limit the movement of the filter such that it is prevented from interfering with or rubbing the bioreactor walls. This is achieved by having some slackness in the tethers connecting the filter to the inner surface of the bioreactor and thus keeping the tethers loose. The degree of slackness in the tethers is such that it allows the filter to move constantly while staying afloat and without touching the inner surface of the bioreactor.

Invention is defined by the claims herein. More advantages and benefits of the present invention will become readily apparent to the person skilled in the art in view of the detailed description below.

### DRAWINGS

The invention will now be described in more detail with reference to the appended drawings, wherein:

FIG. 1A shows a top view of an exemplary filter of the invention;

FIG. 1B shows the various components of the filter illustrated in FIG. 1A;

FIG. 2A shows a top view of the various components of a bioreactor according to a first embodiment of the invention;

FIG. 2B shows a top view of the bioreactor according to the first embodiment as illustrated in FIG. 2A;

FIG. 3A shows a top view of the various components of a bioreactor according to a second embodiment of the invention;

FIG. 3B shows a top view of the bioreactor according to the second embodiment as illustrated in FIG. 3A;

FIG. 4A shows a top view of the various components of a bioreactor according to a third embodiment of the invention;

FIG. 4B shows a top view of the bioreactor according to the third embodiment as illustrated in FIG. 4A;

FIG. 5 shows a side view of an exemplary bioreactor.

### DETAILED DESCRIPTION

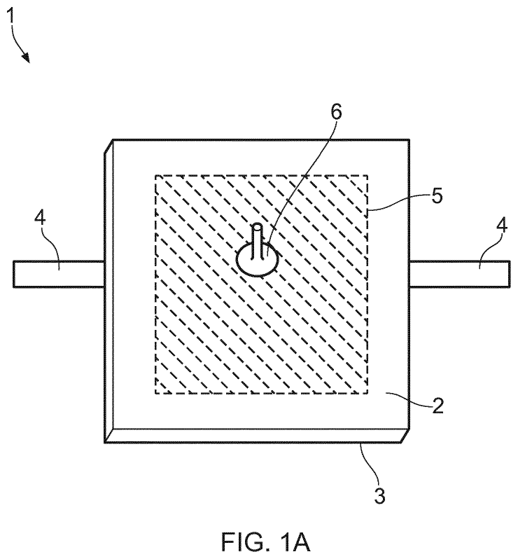

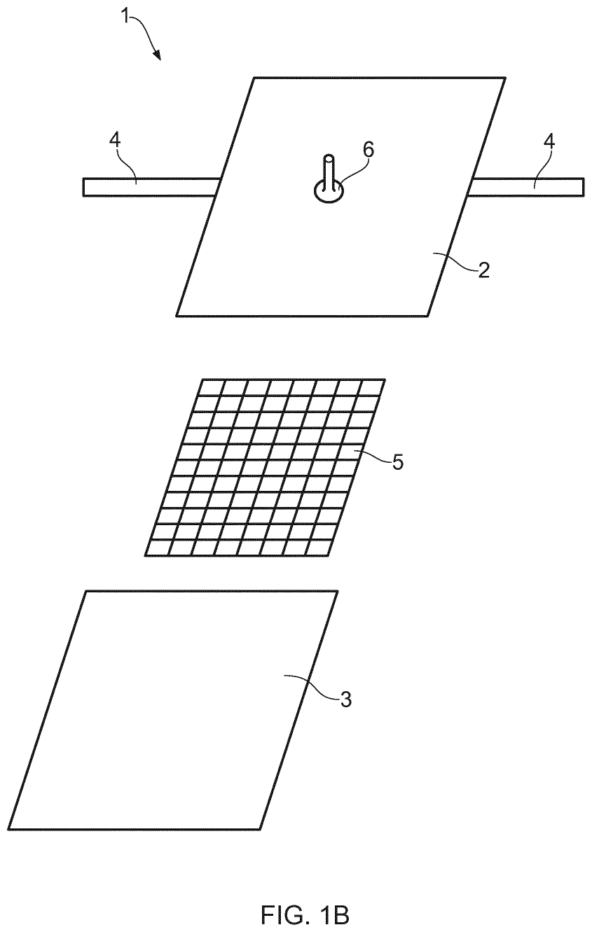

FIG. 1A. shows a top view of an exemplary filter 1 of the invention. The filter 1 comprises a top non-porous layer 2, a bottom filter membrane layer 3 and an open mesh 5 sandwiched between the top layer 2 and the bottom layer 3. The filter 1 also comprises two tethers 4 attached to a surface of the filter 1 to loosely tether the filter 1 to an inner surface of a bioreactor. The filter 1 further comprises a port 6. The port 6 may be connected to a fluid conduit. The fluid conduit may be for example, a flexible plastic or rubber tubing.

FIG. 1B shows the various components of the filter 1 as discussed in the preceding paragraph. FIG. 1B shows the top layer 2 with the tethers 4 attached to a surface of the top layer 2. The top layer 2 also comprises the port 6. FIG. 1B also shows the mesh 5 of the filter 1. The mesh 5 separates the top and bottom layers, providing fluid distribution, and can be made of various materials like, but not limited to Polyethylene terephthalate (PETE). FIG. 1B further shows the bottom layer 3 of the filter 1. The top layer 2 can be made of various materials like, but not limited to Ethylene-vinyl acetate (EVA). The bottom layer 3 can be made of various materials like, but not limited to Ultra-high-molecular-weight polyethylene (UHMWPE), Nylon and Polyethersulfone (PE). The tethers 4 can be made of various materials like, but not limited to EVA. To make the filter 1, the top layer 2 and the bottom layer 3 are sealed together at the edges with the mesh 5 sandwiched between them. The sealing could be done by application of heat, glue or by any other method. In an embodiment of the invention, the top layer 2, the bottom layer 3 and the tethers 4 are made of a flexible heat sealable material such that when heat is applied to the material, it becomes pliable and sticks to itself. The filter 1 is designed such that it allows liquid media and waste metabolites of a cell culture to filter through the bottom filter membrane layer 3 and out through the port 6, via the mesh 5 but does not allow living cells to pass. One way to achieve this is by selecting an appropriate pore size of the bottom filter membrane layer 3. In an alternate design of the filter 1 of the invention, the tethers 4 may be attached to the bottom layer 3 of the filter 1. In yet another design of the filter 1, the tethers 4 may be separate pieces of flexible plastic strips that are heat sealed to a surface of the top layer 2 and/or the bottom layer 3 of the filter 1.

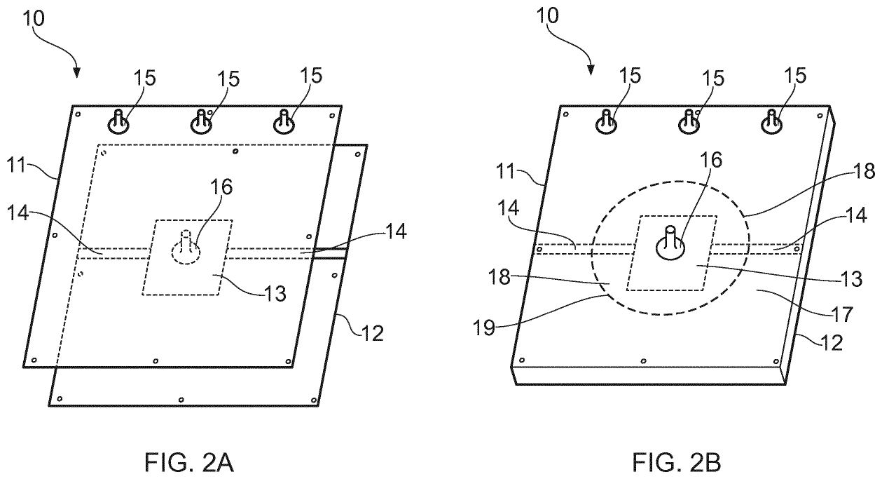

FIG. 2A shows a top view of the various components of a bioreactor 10 according to a first embodiment of the invention. The bioreactor 10 is made of a flexible material and comprises a top layer 11, a bottom layer 12 and a filter 13. The top layer 11 and the bottom layer 12 of the bioreactor can be made of, but not limited to a multilayer laminated clear film of EVA. The top layer 11 comprises three ports 15. The filter 13 comprises a port 16. The ports 15 and 16 facilitate fluidic communication between an internal space and an external space of the bioreactor 10. The filter 13 comprises two tethers 14 attached to a surface of the filter 13 to loosely tether the filter 13 to an inner surface of the bioreactor 10. The top layer 11, the bottom layer 12 and the tethers 14 of the filter 13 are heat sealed to each other such that the filter 13 is situated between the top layer 11 and the bottom layer 12 of the bioreactor 10. The tethers 14 allow limited movement of the filter 13 when the bioreactor 10 is filled at least partially with liquid media.

FIG. 2B shows a top view of the bioreactor 10 according to the first embodiment of the invention as mentioned in the preceding paragraph. The filter 13 is tethered to the inner surface of the bioreactor by the tethers 14. The bioreactor 10 comprises the top layer 11, the bottom layer 12 and the filter 13 situated between the top layer 11 and the bottom layer 12. The tethers 14 of the filter 13 are heat sealed with the top layer 11 and/or the bottom layer 12 such that the bioreactor 10 encloses a bioreactor chamber 17 with the filter 13 situated within the bioreactor chamber 17, the filter 13 being connected to the inner surface of the bioreactor 10 by the tethers 14. When the bioreactor 10 is in use, the filter 13 can only move within the bioreactor chamber 17 within a constrained volume 18. As shown in FIG. 2A, the constrained volume 18 is as enclosed by dotted line 19 and is spaced from the inner surface of the bioreactor 10. As the movement of the filter 13 is constrained, the filter 13 cannot touch or rub against the inner surface of the bioreactor 10. FIG. 2B also shows the three ports on the top layer 11 of the bioreactor 10 and the port 16 on the filter 13. The port 16 can be fluidically connected to the external space of the bioreactor 10 by a fluid conduit. The fluid conduit could be for example, a flexible plastic tubing. A first end of the flexible plastic tubing can be fluidically connected to the port 16 and a second end of the flexible plastic tubing can be connected to a first port 15. This arrangement facilitates transfer of liquid media to the external space of the bioreactor 10 when the bioreactor 10 is used for culturing cells. Similarly, a second port 15 could be used to transfer fresh liquid media from the external space of the bioreactor 10 to the bioreactor chamber 17 of the bioreactor 10. A third port 15 could be connected to an oxygen level sensor.

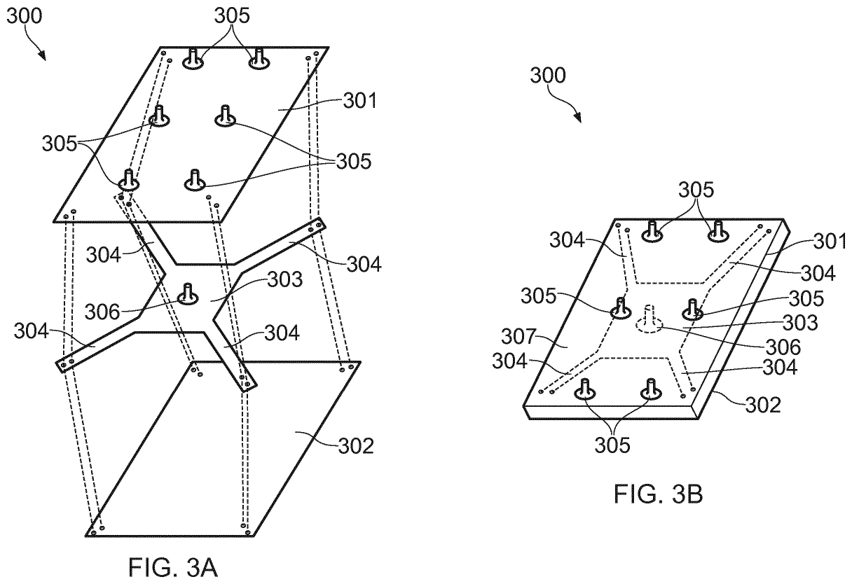

FIG. 3A shows a top view of the various components of a bioreactor 300 according to a second embodiment of the invention. The bioreactor 300 comprises a top layer 301, a bottom layer 302 and a filter 303. The top layer 301 comprises six ports 305. The filter 303 comprises a port 306. The ports 305 and 306 facilitate fluidic communication between an internal space and an external space of the bioreactor 300. The filter 303 comprises four tethers 304 attached to a surface of the filter 303 to loosely tether the filter 303 to an inner surface of the bioreactor 300. The top layer 301, the bottom layer 302 and the tethers 304 are heat sealed to each other along one or more edges such that the filter 303 is situated between the top layer 301 and the bottom layer 302 of the bioreactor 300. The tethers 304 allow limited movement of the filter 303 when the bioreactor 300 is filled at least partially with liquid media. The dotted lines in FIG. 3A show the alignment of the top layer 301, the bottom layer 302, and the filter 303 with the tethers 304 when the bioreactor 300 is assembled during its manufacture.

FIG. 3B shows a top view of the bioreactor 300 according to the second embodiment of the invention as mentioned in the preceding paragraph. The filter 303 is loosely tethered to the inner surface of the bioreactor 300 by the tethers 304. In this embodiment of the invention, the tethers 304 are placed at diagonally opposite corners of the filter 303. The bioreactor 300 comprises the top layer 301, the bottom layer 302 and the filter 303 situated between the top layer 301 and the bottom layer 302. The tethers 304 of the filter 303 are heat sealed with the top layer 301 and/or the bottom layer 302 such that the bioreactor 300 encloses a bioreactor chamber 307 with the filter 303 situated within the bioreactor chamber 307, the filter 303 being connected to the inner surface of the bioreactor 300 by the tethers 304. FIG. 3B also shows six ports 305 on the top layer 301 of the bioreactor 300 and the port 306 on the filter 303. The port 306 can be fluidically connected to the external space of the bioreactor 300 by a fluid conduit. The fluid conduit could be for example, a rubber tubing. A first end of the rubber tubing can be fluidically connected to the port 306 and a second end of the rubber tubing can be connected to a first port 305. This arrangement facilitates transfer of spent liquid media to the external space of the bioreactor 300 when the bioreactor 300 is used for culturing cells. Similarly, a second port 305 could be used to monitor the level of carbon dioxide in a headspace of the bioreactor 300. A third port 305 could be used to introduce fresh liquid media in the bioreactor chamber 307 from an external liquid media source. A fourth port 305 could be used to facilitate gaseous exchange between the bioreactor chamber 307 and the external space of the bioreactor 300. A fifth port 305 could be used to monitor the level of oxygen in the headspace of the bioreactor 300. A sixth port 305 could be connected to a pH sensor.

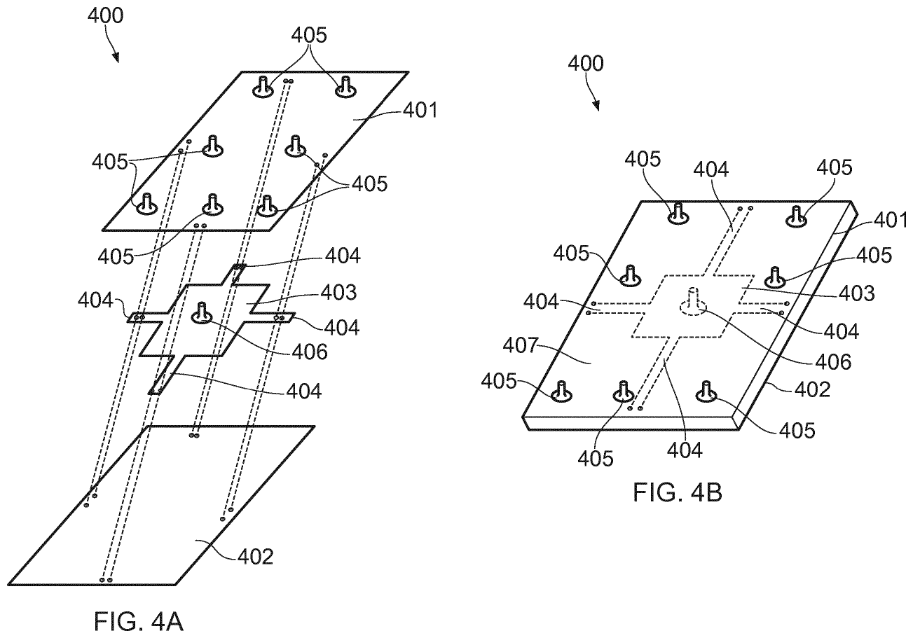

FIG. 4A shows a top view of the various components of a bioreactor 400 according to a third embodiment of the invention. The bioreactor 400 comprises a top layer 401, a bottom layer 402 and a filter 403. The top layer 401 comprises seven ports 405. The filter 403 comprises a port 406. The ports 405 and 406 facilitate fluidic communication between an internal space and an external space of the bioreactor 400. The filter 403 comprises four tethers 404 attached to a surface of the filter 403 to loosely tether the filter 403 to an inner surface of the bioreactor 400. The top layer 401, the bottom layer 402 and the tethers 404 are glued to each other along one or more edges such that the filter 403 is situated between the top layer 401 and the bottom layer 402. The tethers 404 allow limited movement of the filter 403 when the bioreactor 400 is filled at least partially with liquid media. The dotted lines in FIG. 4A show the alignment of the top layer 401, the bottom layer 402, and the filter 403 with the tethers 404 when the bioreactor 400 is assembled during its manufacture.

FIG. 4B shows a top view of the bioreactor 400 according to the third embodiment of the invention as mentioned in the preceding paragraph. The filter 403 is loosely tethered to the inner surface of the bioreactor 400 by the tethers 404. In this embodiment of the invention, the tethers 404 are placed at opposite edges of the filter 403. The bioreactor 400 comprises the top layer 401, the bottom layer 402 and the filter 403 situated between the top layer 401 and the bottom layer 402. The tethers 404 of the filter 403 are glued with the top layer 401 and/or the bottom layer 402 such that the bioreactor 400 encloses a bioreactor chamber 407 with the filter 403 situated within the bioreactor chamber 407, the filter 403 being connected to the inner surface of the bioreactor 400 by the tethers 404. FIG. 4B also shows seven ports 405 on the top layer 401 of the bioreactor 400 and the port 406 on the filter 403. The port 406 can be fluidically connected to the external space of the bioreactor 400 by a fluid conduit. The fluid conduit could be for example, a disposable plastic tubing. A first end of the plastic tubing can be fluidically connected to the port 406 and a second end of the plastic tubing can be connected to a first port 405. This arrangement facilitates transfer of spent liquid media to the external space of the bioreactor 400 when the bioreactor 400 is used for culturing cells. Similarly, a second port 405 could be used to monitor the level of carbon dioxide in a headspace of the bioreactor 400. A third port 405 could be used to introduce fresh liquid media in the bioreactor chamber 407. A fourth port 405 could be used to facilitate gaseous exchange between the bioreactor chamber 407 and the external space of the bioreactor 400. A fifth port 405 could be used to monitor the level of oxygen in the headspace of the bioreactor 400. A sixth port 405 could be connected to a pH sensor. A seventh port 405 could be used to monitor cell density in the bioreactor 400.

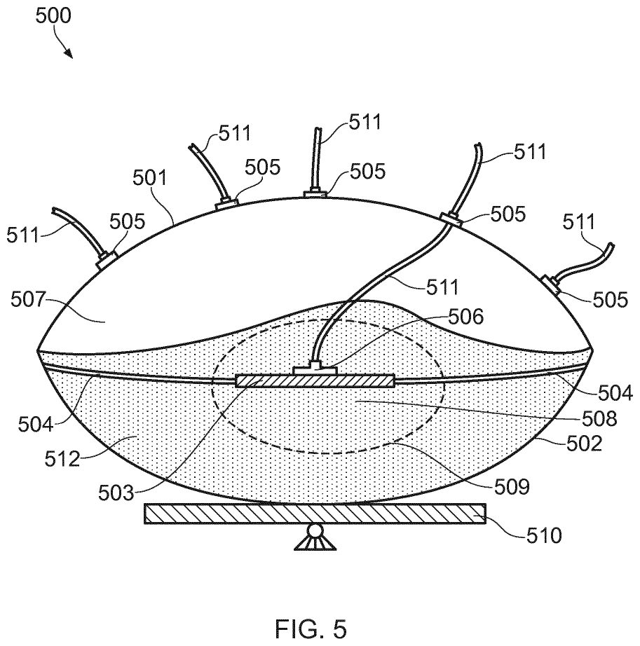

FIG. 5 shows a side view of an exemplary bioreactor 500 with a top layer 501 and a bottom layer 502 enclosing a bioreactor chamber 507 filled partially with liquid media 512. The top layer 501 comprises five ports 505 connected to one or more fluid conduits 511. The ports 505 facilitate fluidic connection between the bioreactor chamber 507 and an external space of the bioreactor 500. The bioreactor 500 also comprises a filter 503 situated within the bioreactor chamber 507. The filter 503 comprises a port 506 and is tethered to an inner surface of the bioreactor 500 by two tethers 504. A first port 505 is fluidically connected to an external liquid media source via a first fluid conduit 511. A second port 505 is fluidically connected to an external seed cells source via a second fluid conduit 511. The port 506 of the filter 503 is fluidically connected to a third port 505 via a third fluid conduit 511. The third port 505 is also connected to an external waste collection bag via a fourth fluid conduit 511. A fourth port 505 is fluidically connected to an external oxygen source via a fifth fluid conduit 511. The bioreactor 500 is placed on a rockable platform 510.

In a method of the invention, the bioreactor 500 is used as a perfusion bioreactor for cell culture and expansion. The bioreactor 500 comprises a disposable cell bag made from flexible plastic material. For example, Xuri bag by GE Healthcare. The bioreactor 500 is filled partially with liquid media 512 via the first port 505 which is fluidically connected to the external liquid media source. The bioreactor 500 is filled with the liquid media such that the filter 503 is afloat on the surface of the liquid media while being at least partially submerged in the liquid media. Seed cells are introduced into the bioreactor chamber 507 of the bioreactor 500 via the second port 505 which is fluidically connected to the external seed cells source. The bioreactor 500 is mounted on top of the rockable platform 510 and the rockable platform 510 is switched on. Rocking of the platform 510 induces wave like motion in the liquid media inside the bioreactor 500. The rockable platform 510 could be for example, GE Healthcare's Wave platform. The rocking motion of the liquid media provides a suitable environment for the seed cells to grow and expand. The rocking motion of the liquid media also induces movement of the filter 503. As the filter 503 is tethered loosely to the inner surface of the bioreactor 500 by tethers 504, the filter 503 is prevented from turning or twisting and stays afloat always while being partially submerged in the liquid media. The tethers 504 constrain the movement of the filter 503 within the bioreactor chamber 507 within a constrained volume 508. As shown in FIG. 5, the constrained volume 508 is as enclosed by dotted line 509 and is spaced from the inner surface of the bioreactor 500. The tethers 504 allow some movement of the filter 503 up and down and laterally such that the filter 503 stays within the constrained volume 508 and hence the filter 504 cannot touch or rub against the inner surface of the bioreactor 500. This prevents damage of the cell bag and the filter 500. This also leads to optimal filtration of the spent liquid media to maintain a healthy growth of the cells in the bioreactor chamber 507. The spent liquid media is filtered through the filter 503 and pumped out via the port 506 to the third port 505 via the third fluid conduit 511.

The spend liquid media is then pumped out of the bioreactor 500 from the third port 505 via the fourth fluid conduit 511 to the waste collection bag.

The invention is not to be seen as limited by the embodiments described above, but can be varied within the scope of the appended claims as is readily apparent to the person skilled in the art. For example, in alternate embodiments, the filter could be attached to the inner surface of the bioreactor by two, three, four or more tethers. The filter could be of any suitable shape including but not limited to a square, a triangle or a circle. The filter could also have more than one ports. The filter could be loosely tethered in various spatial orientations within the bioreactor chamber, with the aim that the filter can move within the bioreactor but not so much that the filter touches the inner surface of the bioreactor. The tethers constrain the movement of the filter but allow some movement of the filter up and down and laterally such that the constrained movement of the filter is within a constrained volume which can be predefined by the amount of slack, flexibility or elasticity of each tether. Ideally the constrained volume is spaced from the inner surface of a cell bag in use to avoid the filter rubbing on the cell bag in use. The constrained volume of the cell bag will change in use as the amount and volume of liquid in the cell bag changes. Thus, at early stages of cell culture process when the cell bag is relatively empty, the constrained volume may be coincident with the inner surface of the cell bag. But, as the liquid volume increases, the cell bag inflates, and the resultant wave motion of that liquid attains more energy during rocking motion of the cell bag, so then the constrained volume of the filter avoids the inner surface of the cell bag to prevent rubbing of the filter with the cell bag at that higher energy phase of the cell culture. In addition, the filter is kept floating on, or immersed in the liquid in the cell bag rather than being lifted by the wave motion of the liquid as rocking of the cell bag proceeds. In a preferred arrangement, the tethers have a length such that there is always some clearance between the filter and the inner surface of the bag, for example at least 10 mm clearance. In practice this can be achieved by multiple tethers working in combination, where at least one will be taut while another is slack at the extremities of the filter's permitted range of movement.

1. A bioreactor comprising:
a bioreactor chamber;
a filter disposed within the bioreactor chamber; and
at least one tether loosely tethering the filter to the bioreactor chamber, the tether(s) allowing the filter to move within the bioreactor chamber within a predefined constrained volume.

2. The bioreactor of claim 1, wherein said constrained volume is spaced from an inner surface of the bioreactor chamber such that the filter cannot touch an inner surface of the bioreactor chamber in use.

3. The bioreactor of claim 1, wherein the bioreactor chamber is coupled to a rocking or rockable platform, the platform being operable such that in use the rocking of the platform induces wave like motion in liquid media within the bioreactor chamber.

4. The bioreactor of claim 1, wherein the filter comprises:
an upper layer;
a filtration membrane coupled to the upper layer; and
the at least one tether is attached to a surface of the filter.

5. The bioreactor of claim 4, wherein the filter is constructed of buoyant material with respect to liquid media within the bioreactor chamber.

6. The bioreactor of claim 4, further comprising a fluid distribution mesh disposed between the upper layer and the filtration membrane of the filter.

7. The bioreactor of claim 1, wherein the filter is fluidically connected to the exterior of the bioreactor chamber through a fluid conduit.

8. The bioreactor of claim 1, wherein the tether(s) is attached to the inner surface of the bioreactor chamber by heat welding.

9. The bioreactor of claim 1, wherein the at least one tether includes a plurality of tethers, each attached to the bioreactor chamber and the filter.

10. The bioreactor of claim 1, wherein the tether(s) comprises or consists of a flexible material.

11. The bioreactor of claim 1, wherein the tether(s) is a flexible plastics strip.

12. A bioreactor system comprising:
a bioreactor chamber in the form of a flexible bag;
a rockable platform supporting the flexible bag in use;
a filter disposed within the bioreactor chamber; and
at least one tether loosely tethering the filter to the bioreactor chamber, the tether(s) allowing the filter to move within the bioreactor chamber in use within a predefined constrained volume.

13. A method of operating a bioreactor comprising:
providing a bioreactor chamber;
providing a filter loosely tethered inside the bioreactor chamber by at least one tether;
at least partially filling the bioreactor chamber with liquid media; and
rocking the bioreactor thereby inducing the filter to move relative to the bioreactor chamber such that the filter moves within the bioreactor chamber within a predefined constrained volume.

14. A method of claim 13, wherein said constrained volume is spaced from an inner surface of the bioreactor chamber such that the filter cannot touch an inner surface of the bioreactor chamber in use, while keeping the filter afloat or partly submerged in liquid media.

15. A method of claim 13, wherein said relative movement induces de-clogging of the filter.

16. The bioreactor of claim 1, wherein the bioreactor chamber is a flexible bag.

17. The bioreactor of claim 1, wherein the bioreactor is a perfusion bioreactor.
